(19) United States
(12) Patent Application Publication
(10) Pub. No.: US 2008/0003192 A1
(43) Pub. Date: Jan. 3, 2008

(54) Functional compositions containing cationic hydrophobically modified polysaccharides for personal care, household & institutional and pet care products

(76) Inventor: Jashawant J. Modi, Hockessin, DE (US)

Correspondence Address: Hercules Incorporated;Hercules Plaza, 1313 North Market Street, Wilmington, DE, 19894-0001, US

(21) Appl. No.: 11/824,317

(22) Filed: Jun. 29, 2007

Related U.S. Application Data
(60) Provisional application No. 60/817,483, filed on Jun. 29, 2006.

(51) Int. Cl. A61K 8/73

(52) U.S. Cl. 424/70.13

(57) ABSTRACT

This invention relates to functional compositions for use in personal care, household & institutional and pet care applications and which contain cationic hydrophobically modified polysaccharides thereby permitting a reduction in the amount of active ingredient material present in the functional composition over amounts typically found in conventional functional compositions while substantially maintaining the level of performance of the functional composition when compared to the level of performance of the conventional functional composition.

### RELATED APPLICATIONS

This application claims the benefit of U.S. Provisional Application Ser. No. 60/817,483, filed on Jun. 29, 2006, which is incorporated herein by reference in its entirety.

### FIELD OF THE INVENTION

The present invention relates to functional compositions useful in personal care, household & institutional and pet care applications. The personal care compositions include, but not limited to, hair care, skin care, sun care, body cleanser, oral care, wipes, and beauty care area. The household & institutional compositions include but are not limited to paper, wood, hard surface substrate cleaners, and auto care products. Pet care compositions include but are not limited to hair care, skin care, sun care, body cleansers, oral care, and beauty care area. Specifically, the personal care and pet care compositions include cleansing, cleaning protecting, depositing, benefiting, moisturizing, firming, conditioning, occlusive barrier, and emollient compositions. The household & institutional care composition more specifically include hand liquid soaps, bar soaps, institution soaps, air fresheners, hand or skin sanitizing liquids, dish washing cleansing wipes, hard surface cleaning wipes and hard surface cleaners

### BACKGROUND OF THE INVENTION

It is known in the personal care, household & institutional and pet care industries to formulate products that provide useful characteristics such as cleansing, cleaning protecting, and benefiting, depositing and moisturizing, and firming, conditioning, providing occlusive barriers, tinting and providing emolliency. Cationic polymers such as Polyquat 10 cationic hydroxyethyl cellulose available from Dow Chemicals, Polyquat 4 cationic hydroxyethyl cellulose available from National Starch, Jaguar™ guar available from Rhodia and N-Hance® cationic guar available from Hercules Incorporated are well known conditioners especially for human and pet hair and skin. However, these current commercial cationic polysaccharides require a higher level of cationic substitution or product use level for them to be effective as a benefiting/conditioning agent. For example, a widely used commercial cationic hydroxyethyl cellulose from Dow Chemicals has cationic degree of substitution as high as 0.4 (UCARE HR400 and JR30M) to be effective as a benefiting/conditioning agent. It is also well known in the industry that this high level of cationic substitution causes undesirable build-up, gumminess, limpness and poor styling of many substrates, for example such as hair. This often situation is often referred to as “over conditioning” It is also well know in the industry that an affinity for cationic polymer increases with increase in the cationic degree of substitution. Reference Amerchol Product Brochure WSP00215192 “UCARE® Polymer LK: A stylish evolution in hair conditioners.” Higher cationic substitution in addition to build-up, causes discoloration or staining of substrates such as textiles, hard surfaces such as bathtubs, wash sinks, ceramic surfaces, porcelain surfaces. None of these commercially available polymers are significantly modified with hydrophobic groups. It was unexpectedly found that cationic hydrophobically modified polysaccharide when incorporated into a functional composition can provide performance equal or superior to similar functional compositions containing current commercially available cationic polysaccharides.

Many polysaccharides are also used as rheology modifiers for personal care, household & institutional and pet care products. Polysaccharide derivatives such as cellulose ethers, and polygalactomannan and polygalactomannan derivative products are among the best-known polysaccharides for use as rheology modifiers.

Various polysaccharides have been taught as having use in various end-use applications. U.S. Pat. No. 4,663,159 discloses hydrophobe substituted, water-soluble cationic polysaccharides and their use in personal care, emulsions and cleaners. U.S. Pat. No. 4,001,394 discloses a shampoo creme rinse. U.S. Pat. No. 5,407,919 discloses double substituted water-soluble cellulose ethers where the cellulose ether is substituted with a cationic substituent and a hydrophobic substituent and further discloses the use of this compound in personal care applications. U.S. Pat. No. 6,372,901 discloses an alkyl-aryl hydrophobically modified hydroxyethyl cellulose useful as an associative thickener in paints. WO 2005/000903 A1, discloses cellulose ethers comprising a hydrophobic substituent and a cationic substituent and further discloses the use of this material in personal care applications.

The need still exists for functional compositions having less active ingredient than the levels of ingredients presently used while still providing an equivalent level of functionality. Functional compositions with less active ingredients may reduce some of the negative aspects associated with active ingredients, such as the harshness of surfactants in personal care applications, while still providing an adequate level of cleaning may be beneficial.

### SUMMARY OF THE INVENTION

The present invention is directed to a functional composition useful for cleansing, cleaning, protecting, benefiting, depositing, moisturizing, firming, conditioning, providing occlusive barrier, or providing an emollient. The functional composition comprising a) at least about 0.01 wt % of a cationic hydrophobically modified polysaccharide wherein i) the cationic hydrophobic polysaccharide has a mean average molecular weight (Mw) from about 2000 to about 10,000,000 Dalton, ii) the cationic hydrophobically modified polysaccharide has a cationic degree of substitution greater than 0.001 to about 3.0, and iii) cationic hydrophobically modified polysaccharide has weight % hydrophobe greater than 0.001 and b) less than 50.0 wt % total of a personal care, pet care or household & institutional care active ingredient material.

Examples of the personal care active ingredient materials include sun ray (UV) absorbers, sun screen agents, moisturizers, humectants, benefiting agents for hair, skin, nails and mouth, depositing agents such as surfactants and polysaccharide polymers, occlusive agents, moisture barriers, lubricants, emollients, anti-aging agents, antistatic agents, abrasive, antimicrobial agents, insect repellent agents, drug delivery agents, secondary conditioners, exfoliants, lustering agents, tanning agents, luminescent, colors, anti-odorizing agents, fragrances, viscosifying agents, salts, lipids, phospholipids, hydrophobic plant extracts, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons, and mixtures thereof.

Examples of the household & institutional active ingredient materials include dust collectors, polishing agents, stain removing agents, anti-redeposition agents, coloring agents, tinting agents, scrubbing agents, UV absorbers, sun screen agents, moisturizers, humectants, benefiting agents for wood, tiles, and other hard surfaces, automobile treatment agents, lubricants anti-aging agents, antistatic agents, abrasives, antimicrobial agents, drug delivery agents, secondary conditioners, exfolliants, lustering agents, luminescent, anti-odorizing agents, fragrances, viscosifying agents, salts, lipids, phospholipids, hydrophobic plant extracts, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons, and mixtures thereof.

Examples of the pet care active ingredient materials include uv absorbers, sun screen agents, moisturizers, humectants, benefiting agents for hair, skin and mouth, deposition agents, occlusive agents, moisture barriers, lubricants, emollients, anti-aging agents, antistatic agents, abrasives, antimicrobial agents, insect repellent agents, drug delivery agents, secondary conditioners, exfolliants, lustering agents, tanning agents, luminescent, colors, anti-odorizing agents, fragrances, viscosifying agents, salts, lipids, phospholipids, hydrophobic plant extracts, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons, and mixture thereof.

### DETAILED DESCRIPTION OF THE INVENTION

In accordance with the invention, the cationic hydrophobically modified polysaccharide of use in the functional composition has i) a mean average molecular weight (Mw) from about 2000 to about 10,000,000 Dalton, preferably from about 50,000 to about 2,000,000 Dalton, more preferably from about 200,000 to about 1,000,000 Dalton; ii) a cationic degree of substitution greater than 0.001 to about 3.0, preferably greater than 0.05 to about 2.0, more preferably greater than 0.1 to about 1.5, more preferably greater than 0.2 to about 1.0; and iii) a weight % hydrophobe greater than 0.01%, preferably in the range of 0.1 to about 5%, more preferably in the range of 0.2 to about 3%, still more preferably in the range of 0.25 to about 1.5%. Being hydrophobic, these cationic hydrophobically modified polysaccharide polymers provide equal or better conditioning even with a lower cationic substitution than commercially available cationic polysaccharides. Application of these cationic hydrophobically modified polysaccharide polymers in functional compositions of this invention is in personal care, household & institutional and pet care products.

One advantage of the use of cationically hydrophobically modified polysaccharide polymers in functional compositions is that the amount of active ingredient material needed for a functional composition is reduced for compositions of equivalent functionality. The total amount of active ingredient material found in the functional composition of the invention is less than 50 wt % of the functional composition, preferably less than 30 wt %, more preferably less than 10 wt %, still more preferably less than 5 wt % and still more preferably less than 1 wt %. The total amount of active ingredient material found in the functional composition of the invention may be in the range of from 0.01 to 50 wt %, more preferably in the range of from 0.01 to 30 wt %, still more preferably in the range from 0.5 to 15 wt %.

A cationic hydrophobically modified polysaccharide of use in the present invention is more fully described in U.S. patent Ser. No. 11/353,621, incorporated herein by reference in its entirety.

Other cationically hydrophobically modified polysaccharides which may be of use in the present invention are more fully described in U.S. Pat. Nos. 5,407,919 and 6,372,901, incorporated herein by reference in their entireties and patent application WO2005000903, incorporated herein by reference in its entirety.

In the functional compositions of this invention, the cationic hydrophobically modified polysaccharides contained therein allow users to reduce surfactant level, which are generally considered irritant and harsh for skin, hair, and scalp of both human and animals, while maintaining desired properties such as lather richness, feel, volume and stability. The functional compositions of the invention also reduce or eliminate the need for inorganic and/or organic salts which are commonly used as viscosifying agents. These salts are also considered irritants which can leave skin and hair with a dry rough feel. In addition, the cationic hydrophobically modified polysaccharide polymer due to its positive charge has affinity for negatively charged surfaces.

Personal care compositions include hair care, skin care, sun care, nail care, and oral care compositions. Examples of active substances that may suitably be included, but not limited to, in the personal care products according to the present invention are as follows:

1) Perfumes, which give rise to an olfactory response in the form of a fragrance and deodorant perfumes, which in addition to providing a fragrance response can also reduce body malodor;

2) Skin coolants, such as menthol, menthyl acetate, menthyl pyrrolidone carboxylate N-ethyl-p-menthane-3-carboxamide and other derivatives of menthol, which give rise to a tactile response in the form of a cooling sensation on the skin;

3) Emollients, such as isopropylmyristate, silicone materials, mineral oils and vegetable oils which give rise to a tactile response in the form of an increase in skin lubricity;

4) Deodorants other than perfumes, whose function is to reduce the level of or eliminate micro flora at the skin surface, especially those responsible for the development of body malodor. Precursors of deodorants other than perfume can also be used;

5) Antiperspirant actives, whose function is to reduce or eliminate the appearance of perspiration at the skin surface;

6) Moisturizing agents, that keep the skin moist by either adding moisture or preventing from evaporating from the skin;

7) Cleansing agents, that remove dirt and oil from the skin;

8) Sunscreen active ingredients that protect the skin and hair from UV and other harmful light rays from the sun. In accordance with this invention a therapeutically effective amount will normally be from 0.01 to 10% by weight, preferable 0.1 to 5% by weight of the composition;

9) Hair treatment agents, that condition the hair, cleanse the hair, detangles hair, acts as styling agent, volumizing and gloss agents, color retention agent, anti-dandruff agent, hair growth promoters, hair dyes and pigments, hair perfumes, hair relaxers, hair bleaching agent, hair moisturizer, hair oil treatment agent, and antifrizzing agent;

10) Oral care agents, such as dentifrices and mouthwashes, that clean, whiten, deodorize and protect the teeth and gum;

11) Denture adhesives that provide adhesion properties to dentures;

12) Shaving products, such as creams, gels and lotions and razor blade lubricating strips;

14) Beauty aids, such as foundation powders, lipsticks, and eye care;

15) Textile products, such as moisturizing or cleansing wipes;

16) Liquid and gels, which are used to cleaning animal teeters; and

17) Pet hair and skin care products used for keeping animal free of soils, unwanted insects and to protect hair and skin from undesirable environments,

In accordance with the present invention, when the functional system is a household & institutional care & institutional compositions, this household & institutional care product includes a cationic hydrophobically modified polysaccharide and at least one active household & institutional care ingredient. Examples of active substances that may suitably be included, but not limited to, according to the present invention are as follows:

1) Perfumes, which give rise to an olfactory response in the form of a fragrance and deodorant perfumes, which in addition to providing a fragrance response can reduce odor;

2) Insect repellent agent whose function is to keep insects from a particular area or attacking skin;

3) Bubble generating agent, such as surfactant that generates foam or lather;

4) Industrial grade bar, shower gel, and liquid soap actives that remove germs, dirt, grease and oil from skin, sanitizes skin, and conditions the skin;

5) All purpose cleaning agents, that remove dirt, oil, grease, and germs from the surface in areas such as kitchens, bathroom, and public facilities;

6) Disinfecting ingredients that kill or prevent growth of germs in a house or public facility;

7) Dishwashing detergents, which remove stains, food, germs;

8) Toilet bowl cleaning agents, which remove stains, kills germs, and deodorizes;

9) Vehicle cleaning actives, which remove dirt, grease, etc. from vehicles and equipment; and

10) Lubricating agent that reduces friction between parts.

Among the household & institutional care products which the functional system of the present invention may be of use are:

1) Textile products, such as dusting or disinfecting wipes;

2) Toilet Care Wipes—adult and baby wet wipes used for removing undesirable soil;

3) Dry Wipes—(dry wipes containing polymer invention and other actives such as surfactant. antimicrobial agent, emollient, moisturizer) wherein the wipes or a substrate to which the wipes are applied is prewetted with water or a water containing vehicle prior to use;

4) Dry polymer films containing ingredients such as surfactant, antimicrobial agent emollient, moisturizer wherein the film or a substrate for which the dry polymer film are to be used is prewetted with water or water containing vehicle prior to use;

5) Dishwashing wipes used for cleaning kitchen pots, pan, dishes, and utensils;

6) Bath room wipes used for cleaning bathrooms;

7) Furniture wipes used for cleaning upholstered and upholstered furniture;

8) Hard surface cleaning wipes used for kitchen counter, bathroom counters, and floors;

9) Institution wipes used in hospital, restaurants, and commercial buildings;

10) All purpose cleaning wipes used for general cleaning of various surfaces;

11) Deodorant wipes used for deodorizing surfaces;

12) Personal lubricants in liquid or gel form used by hospitals and health care professionals for reducing friction in procedures such as for example reducing friction between a patient's skin and an ultrasound transducer probe.

In accordance with the present invention, when the functional system is a pet care composition, this pet care composition includes a cationic hydrophobically modified polysaccharide and at least one active pet care ingredient. Examples of active pet care ingredients that may suitably be included, but not limited to, according to the present invention are as follows:

1) Perfumes, which give rise to an olfactory response in the form of a fragrance and deodorant perfumes, which in addition to providing a fragrance response can also reduce body malodor;

2) Skin coolants, such as menthol, menthyl acetate, menthyl pyrrolidone carboxylate N-ethyl-p-menthane-3-carboxamide and other derivatives of menthol, which give rise to a tactile response in the form of a cooling sensation on the skin;

3) Emollients, such as isopropylmyristate, silicone materials, mineral oils and vegetable oils which give rise to a tactile response in the form of an increase in skin lubricity;

4) Deodorants other than perfumes, whose function is to reduce the level of or eliminate micro flora at the skin surface, especially those responsible for the development of body malodor. Precursors of deodorants other than perfume can also be used;

6) Moisturizing agents, that keep the skin moist by either adding moisture or preventing from evaporating from the skin;

7) Pet hair cleansing agents, that remove insect, soil, dirt and oil from the hair;

8) Pet skin cleansing agents, that remove insect, soil, dirt and oil from the skin;

9) Sunscreen active ingredients that protect the skin and hair from UV and other harmful light rays from the sun. In accordance with this invention a therapeutically effective amount will normally be from 0.01 to 10% by weight, preferable 0.1 to 5% by weight of the composition;

10) Hair treatment agents, that condition the hair, cleanse the hair, detangles hair, acts as styling agent, volumizing and gloss agents, color retention agent, anti-dandruff agent, hair growth promoters, hair dyes and pigments, hair bleaching agent, hair moisturizer, hair oil treatment agent, and antifrizzing agent;

11) Oral care agents, such as dentifrices and mouthwashes, that clean, whiten, deodorize and protect a pet's teeth and gums;

12) Care wipes to keep pet clean, deodorized and to protect from unwanted soil, contaminations, and insects.

The above lists of personal care and household & institutional care and pet care active ingredients are only examples and are not complete lists of active ingredients that can be used. Other ingredients used in these types of products are well known in the industry.

The following examples will serve to illustrate the invention, all parts and percentages being by weight, unless otherwise indicated.

### EXAMPLES

Cationic hydrophobically modified polysaccharides of use in the present invention were made in the following manner:

### Cationic hydrophobically modified hydroxyethyl cellulose—CatC16HMHEC

To a stirred vessel, the following was added.

16.2 kg lb of tert-butanol, 1077 grams of isopropanol, 703 grams of caustic soda (50 wt % pure) and 1878 grams of water. This mixture was cooled to 25° C. and stirred for 30 minutes.

After this period, 2100 grams of cellulose was added. The reactor was sealed and nitrogen was purged through to remove oxygen. This mixture was stirred for 45 minutes at 25° C.

After 45 minutes, 1995 grams of ethylene oxide was added. The pressure was increased to 35 psig with nitrogen.

The reactor mass was heated to 95° C. and held at this temperature for 60 minutes. After cooling to 55° C., the reactor was vented to atmospheric pressure, and 933 grams of nitric acid (69.5% purity) was added. The mixture was stirred for 5 minutes. Next, 555 grams of hexadecylglycidylether was added. The reactor was closed; the pressure raised to 35 psig with nitrogen. After a two minute hold period, the pressure was released. This was repeated two more times.

Ethylene oxide was added (1466 grams). The pressure inside the reactor was increased to 35 psig with nitrogen.

The reactor mass was heated to 120° C. and held at this temperature for 3 hours. After this period, the mixture was cooled to 55° C. The pressure was released, and 798 grams of a 69 wt % aqueous solution of 3-chloro-2-hydroxypropyltrimethylammonium chloride in water was added.

Vacuum was applied for two minutes, and then the pressure was increased with nitrogen to about 25 psig. The reactor mass was heated to 60° C. and held at this level for one hour.

After the reaction was completed, the mass was cooled, the pressure was released and 339 grams of nitric acid (69.5% purity) was added.

After the acid was mixed well with the contents of the reactor, the mixture was removed from the reactor.

The tert-butanol solvent mix was removed by means of syphonation.

A mixture of 10 vol % water and 90 vol % acetone was added, and the polymer was purified with this for 15 minutes. After this mixing period, the acetone was removed. A fresh amount of 90/10 vol % acetone/water mixture was added, and mixed for 15 minutes, after which the acetone mixture was removed. This was repeated twice more.

The final dehydration of the polymer was done in a similar way as the purification, with the exception that 100% pure acetone was used. The polymer was dehydrated by means of two washes with pure acetone.

The polymer was transferred in to a vacuum oven, and dried until the moisture content was below 8 wt %.

The final polymer had an HE-MS of 3.81, a Cat-DS of 0.088 and a hexadecyl substitution level of 1.14 wt %.

### Cationic Hydrophobically Modified Hydroxyethyl cellulose—CatC8HMHEC

To a stirred vessel, the following was added:

17.9 kg lb of tert-butanol, 1229 grams of isopropanol, 686 grams of caustic soda (50 wt % pure) and 1339 grams of water. This mixture was cooled to 25° C. and stirred for 30 minutes.

After this period, 2270 grams of cellulose were added. The reactor was sealed and nitrogen was purged through to remove oxygen. This mixture was stirred for 45 minutes at 25° C.

After 45 minutes, 144 grams of octylbromide and 1768 grams of ethylene oxide were added. The pressure was increased to 35 psig with nitrogen.

The reactor mass was heated to 55° C. and held at this temperature for 30 minutes. Next, the mixture was heated to 95° C., held for 15 minutes and then heated to 125° C. and held for 60 minutes. After cooling to 35° C., the reactor was vented to atmospheric pressure, and 946 grams of nitric acid (69.5% purity) was added. The mixture was stirred for 5 minutes. Next, 182 grams of a 69 wt % aqueous solution of 3-chloro-2-hydroxypropyltrimethylammonium chloride in water was added.

Vacuum was applied for two minutes, and then the pressure was increased with nitrogen to about 25 psig. The reactor mass was heated to 60° C. and held at this level for one hour.

After the reaction was completed, the mass was cooled, the pressure was released and 411 grams of nitric acid (69.5% purity) were added.

After the acid was mixed well with the contents of the reactor, the mixture was removed from the reactor.

The tert-butanol solvent mix was removed by means of syphonation.

A mixture of 10 vol % water and 90 vol % acetone was added, and the polymer was purified with this for 15 minutes. After this mixing period, the acetone was removed. A fresh amount of 90/10 vol % acetone/water mixture was added, and mixed for 15 minutes, after which the acetone mixture was removed. This was repeated twice more.

The final dehydration of the polymer was done in a similar way as the purification, with the exception that 100% pure acetone was used. The polymer was dehydrated by means of two washes with pure acetone.

The polymer was transferred in to a vacuum oven, and dried until the moisture content was below 8 wt %.

The final polymer had an HE-MS of 2.20 a Cat-DS of 0.040 and an octyl substitution level of 0.80 wt %.

### Example 1

### Personal Care

Hair Conditioner: The functional compositions of the invention were formulated into a hair conditioning formulation to evaluate for their wet and dry combing performance.

| | |
|---|---|
| 90.94 g | Deionized water |
| 00.70 g | Natrosol ® 250HHR hydroxyethyl cellulose |
| 00.20 g | Cationic hydrophobically polysaccharide or |
| | commercial polymer |
| 02.00 g | Cetyl alcohol |
| 00.50 g | Potassium Chloride |
| 02.00 g | Isopropyl Myristate |
| As required | citric acid to adjust pH |
| As required | Sodium hydroxide to adjust pH |
| 00.50 g | Preservative, Germaben ® II preservative (available |
| | from International Specialty Products) |

The conditioner formulation was prepared by first adding Natrosol® hydroxyethyl cellulose type 250HHR (available from Hercules Incorporated) to water under agitation to form a slurry. Next, pH of the slurry was adjusted to 8.0 to 8.5. The slurry was stirred for about 30 minutes or until polymer fully dissolved. Next, a cationic hydrophobically modified polysaccharide polymeror a comparative polymer listed in Table 1 was added and mixed for 30 more minutes. The solution was heated to about 65° C. and stirred until it became smooth. Next, cetyl alcohol was added and mixed until it appeared homogeneously mixed. The mixture was cooled to about 50° C. while mixing and then potassium chloride was added. Next, isopropyl myristate was added mixed until the mixture looked homogeneous. The mixture pH was adjusted between 5.25 and 5.5 with citric acid and/or NaOH solution. Next, the conditioner was preserved with 0.5% preservative and mixing was continued until it reached room temperature. The conditioner was used for its combing performance. All conditioners contained 0.7% Natrosol® 250HHR hydroxyethyl celluloseas thickener. All polymers of this invention and comparative polymers were used at 0.2% level.

To test the conditioner for combing, one inch wide flat tresses of mildly bleached European hair weighing about 3 grams were used for the study. The hair tresses were obtained from International Hair Importers and Products Inc. of Glendale, N.Y. The hair tresses were first cleaned with 4.5% active sodium lauryl sulfate solution. To clean the hair tress, the hair tress was first wetted with 40° C. tap water and then 5.0 ml of sodium lauryl sulfate solution was applied along the tress length. Tress was kneaded for 30 second. Tress was then rinsed under 40° C. running water for 30 seconds followed by rinsing with room temperature tap water for 30 seconds. The tress was then dried overnight.

Next day tress was rewetted with 40° C. tap water and then 0.5 gram conditioner per gram of hair was applied uniformly along the length of hair. Tress was kneaded for 30 second and then it was rinsed under 40° C. running water for 30 seconds. Same amount of conditioner was reapplied again along the length of the tress, tress was kneaded for 30 seconds, and then it was rinsed under 40° C. running water for 30 seconds. The tress was rinsed with room temperature tap water for 30 seconds. Tress was combed immediately for eight times on Instron testing machine to measure wet combing energy (gf-mm/g) to required comb. From the data, average wet combing energy in gram force-mm/gram of hair (gf-mm/g) was calculated. The tress was then stored overnight at about 50% relative humidity and about 23° C.

Next day, tress was first combed with fine teeth rubber comb to free-up hair stuck together. Again, hair tress was combed eight times on Instron testing machine to determine average force required to comb one gram of dry hair. A higher the value the poorer the conditioning effect of the polymer being tested. Two tresses were used per conditioning formulation. The data reported in Table 1 are average of two tresses.

TABLE 1

| | Cationic | | | Conditioner | | | |
|---|---|---|---|---|---|---|---|
| | hydrophobically | Commercial and other | Polymer | Viscosity | Wet Combing | Dry Combing | |
| ID # | polysaccharide | polymers | Level | (cps) | (gf-mm/g) | (gf-mm/g) | Comments |
| 1-1 | | Control - with 250HHR @ 0.7% | 0 | 990 | 4,774 | 287 | Stable |
| 1-2 | | Control - with 250HHR @ 0.7% | 0 | 1,380 | 4,513 | 364 | Stable |
| 1-3 | | N-Hance ® 3269 | 0.2 | 1,330 | 1,389 | 263 | Stable |
| 1-4 | | AquaCat ® CG518 | 0.2 | 1,100 | 1,142 | 332 | Stable |
| 1-5 | | Natrosol ® 250HHR | 0.2 | 1,970 | 4,320 | 361 | Stable |
| 1-6 | | Natrosol 250HHR | 0.2 | 2100 | 2,700 | 290 | Stable |
| 1-7 | | UCARE ® LR400 | 0.2 | 1280 | 811 | 1116 | Stable |
| 1-8 | | UCARE ® JR30M | 0.2 | 1,960 | 868 | 849 | Stable |
| 1-9 | | Nexton ® 3082R | 0.2 | 2,280 | 4,941 | 312 | Stable |
| 1-10 | | Natrosol ® Plus 330 | 0.2 | 1,670 | 2,565 | 340 | Stable |
| 1-11 | | Polysurf 67 | 0.2 | 2,170 | 2,952 | 459 | Stable |
| 1-12 | | C8HMHEC | 0.2 | 1,080 | 2,281 | 625 | Stable |
| 1-13 | | C16 HMHEC | 0.2 | 1,940 | 2,262 | 298 | Stable |
| 1-14 | Cat C8HMHEC | | 0.2 | 1,310 | 916 | 393 | Stable |
| 1-15 | Cat C16 HMHEC | | 0.2 | 1,050 | 751 | 546 | Stable |

Examples 1-14 and 1-15 represents functional compositions of invention. Example 1-1 to 1-13 is comparative examples.

In a conditioner formulation, the cationic hydrophobically modified polysaccharide polymer—Cat C16 HMHEC provides better wet combing than the corresponding nonionic C16 HMHEC, commercial Natrosol Plus 330CS hydroxyethyl cellulose and Polysurf® 67 cetyl hydroxyethyl cellulose (both available from Hercules Incorporated). It also provided wet combing equal to commercial cationic HECs (UCARE® cationic hydroxyethyl cellulose available from Dow Chemicals) but dry combing better than the cat HECs.

Cationic C8HMHEC provides better wet and dry combing than the nonionic CBHMHEC. It also provides wet combing equal to commercial cationic HECs (UCARE® cationic hydroxyethyl cellulose) but better dry combing than the commercial cationic HECs.

The polymers of invention also had better balance of wet combing than the commercial cationic guars—N-Hance® guar and AquaCat® CG 518 guar, both available from Hercules Incorporated.

### Ingredient List for Table 1:

### (1) Natrosol® 250HHR: Hydroxyethyl cellulose from Hercules Incorporated Wilmington, Del.

### (2) Nexton® 3082R: C4 hydrophobically modified hydroxyethyl cellulose from Hercules Incorporated Wilmington, Del.

### (3) Polysurf® 67C16 hydrophobically modified hydroxyethyl cellulose from Hercules Incorporated

### (4) Natrosol® Plus 330C16 hydrophobically modified hydroxyethyl cellulose from Hercules Incorporated

### (5) UCARE® LR400 cationic HEC from Dow Chemicals, Midland, Mich.

### (6) UCARE® JR30M cationic HEC from Dow Chemicals, Midland, Mich.

### (7) N-Hance® 3269 cationic guar cationic DS 0.13, Weight average Molecular weight 500,000 from Hercules Incorporated, Wilmington, Del.

### (8) AquaCat® CG 518 cationic guar, cationic DS 0.18, Weight average Molecular weight 50,000 from Hercules Incorporated, Wilmington, Del.

### (9) C8 hydrophobically modified hydroxyethyl cellulose from Hercules Incorporated.

### (10) Cationic hydrophobically modified polysaccharide polymer, Cationic degree of substitution=0.029, weight % C8=0.64. Cationic C8 hydrophobically modified hydroxyethyl cellulose from Hercules Incorporated

### (11) Cationic hydrophobically modified polysaccharide polymer, cationic degree of substitution=0.08, Weight % C16=1.48. Cationic C16 hydrophobically modified hydroxyethyl cellulose from Hercules Incorporated

### (12) C16 hydrophobically modified hydroxyethyl cellulose from Hercules Incorporated

(13) Crodacol C95NF cetyl alcohol from Croda Inc. Parsippany, N.J.

### (14) KCl: Potassium chloride

### (15) Stepan® IPM, Isopropyl myristate from Stepan Company, Northfield, Ill.

### (16) Germaben® II preservative from ISP Wayne, N.J.

### Example 2

### Personal Care

Hair Conditioner The conditioners in Table 2 were prepared and the tested for combing the same way as described in Example 1. Again all conditioner had 0.7% Natrosol® 250HHR hydroxyethyl cellulose, available from Hercules Incorporated as thickener.

TABLE 2

| | Cationic hydrophobically | Commercial and other | Polymer | Viscosity | Wet Comb Energy | Dry Comb Energy | |
|---|---|---|---|---|---|---|---|
| ID # | modified polysaccharide | polymers | Level | (cps) | (gf-mm/g) | (gf-mm/g) | Comments |
| 2-1 | | Control - with Natrosol ® | 0.0 | 910 | 5,497 | 745 | creaming |
| | | 250HHR HEC @ 0.7% | | | | | |
| 2-2 | | Natrosol ® 250HHR HEC | 0.2 | 1,850 | 6,164 | 573 | Stable |
| 2-3 | | N-Hance ® 3215 | 0.2 | 1,630 | 1,646 | 297 | Stable |
| 2-4- | Cat C4HMGuar | | 0.2 | 1,290 | 2,481 | 329 | Stable |
| 2-5 | Cat C4HMHEC | | 0.2 | 1,460 | 1,228 | Could not comb. | Stable |
| | | | | | | Entangled | |
| | | | | | | at tip | |
| 2-6 | | UCARE ® JR30M | 0.2 | 2,092 | 1,106 | Could not comb. | Stable |
| | | | | | | Entangled | |
| | | | | | | at tip | |
| 2-7 | | Nexton 3082R | 0.2 | 2,232 | 7,379 | 456 | Stable |
| 2-8 | | N-Hance HP40 | 0.2 | 1,968 | 6,433 | 529 | Stable |
| | | K2140B | | | | | |
| 2-9 | | Jaguar HP105 | 0.2 | 2,600 | 5,162 | 388 | Stable |
| 2-10 | Cat C16HMHEC | | 0.2 | 1,532 | 1,423 | 214 | Stable |
| 2-11 | | SoftCat SL60 | 0.2 | 2,260 | 1,374 | Entangled | Stable |

Example 2-4, 2-5 and 2-10 represents cationic hydrophobically modified polysaccharide polymer. Example 2-1, 2-3, 2-6 to 2-9 and 2-11 represents comparative examples.

The cationic hydrophobically modified polysaccharide polymer Cat C16HMHEC of use in the functional compositions of this invention provided better wet and dry combing than the commercial mix quat HMHEC (SoftCat® SL60) from Dow Chemicals and hydroxypropylated guar (Jaguar® HP105) from Rhodia. It also provided much better dry combing than the commercial cationic HEC (UCARE® JR30M) from Dow Chemicals.

The cationic hydrophobically modified polysaccharide polymer Cat C4HMHECs provided good wet combing than the commercial nonionic C4HMHEC (Nexton®) from Hercules Incorporated.

Ingredient List for Table 2:

(1) Crodacol C95NF cetyl alcohol from Croda Inc. Parsippany, N.J.

### (2) KCl: Potassium chloride

### (3) Stepan® IPM Isopropyl myristate from Stepan Company, Northfield, Ill.

### (4) Germaben® II preservative from ISP Wayne, N.J.

### (6) N-Hance® 3215, cationic guar, Hercules Incorporated, Wilmington, Del.

### (7) Cationic DS=0.42, Molecular weight 1.4 million. cationic hydrophobically modified polysaccharide polymer, C4 Hydrophobically modified Cationic guar Hercules Incorporated, Wilmington, Del.

(8) Cationic hydrophobically modified polysaccharide polymer, Wt % C4HM 1.59%, Cationic charge density 1.30. Cat. C4 Hydrophobically modified Hydroxyethyl cellulose Hercules Incorporated Wilmington, Del.

### (9) UCAREG JR30M, cationic HEC from Dow Chemicals, Midland, Mich.

### (10) Nexton® 3082R: hydrophobically modified hydroxyethyl cellulose from Hercules Incorporated, Wilmington, Del.

### (11) Natrosol® 250HHR hydroxyethyl cellulose from Hercules Incorporated, Wilmington, Del.

### (12) N-Hance® HP40; hydroxypropyl guar, Hercules Incorporated, Wilmington, Del.

### (13) Jaguar HP105 hydroxypropyl guar, HP MS 0.71, Molecular weight 4,850,000, aqueous Brookfield viscosity 3076 cps at 1.0% Rhodia; Cranbury, N.J.

(14) Cationic C16 HMHEC. 0.94 wt % cetyl, Cationic DS 0.080, aqueous Brookfield viscosity 1620 cps. At 1.0%, 30 rpm
(15) SoftCat® SL60: Mixed quat hydrophobically modified hydroxyethyl cellulose. The Dow Chemical Company, Midland Mich.

### Example 3

### Personal Care

### Skin Lotion

The control skin lotion formulation was made with 0.5% Natrosol® Plus 330 hydroxyethyl cellulose. In this experiment, Natrosol® Plus 330 hydroxyethyl cellulose was replaced with cationic hydrophobically modified polysaccharide polymer or comparative polymers listed in Table 3.

| Ingredient | | Weight % Active |
|---|---|---|
| A. | Natrosol ® Plus CS HEC, Grade 330 | 0.50 |
| | Distilled water | 78.00 |
| | Glycerin, | 2.00 |
| B. | Glycol stearate (Kessco ® EGMS) | 2.75 |
| | Stearic acid (Industrene ® 5016) | 2.50 |
| | Mineral oil (Drakeol ® 7) | 2.00 |
| | Acetylated lanolin (Lipolan ® 98) | 0.50 |
| | Cetyl alcohol (Crodacol ® C95) | 0.25 |
| C. | Distilled water | 10.00 |
| | Triethanolamine | 0.50 |
| D. | Propylene glycol and diazolidinyl urea | 0.75 |
| | and methylparaben and propylparaben | |
| | Germaben ® II preservative | |
| | | 100.00 |

Polymer listed in Table 3 was dispersed in water by adding to the vortex of well-agitated from Part A. It was mixed for five minutes. Next, glycerin was added with continued mixing and heated to 80° C. Mixed 15 minutes at 80° C. In a separate vessel, blended Part B ingredients and heated to 80° C. and mixed well. Added Part B to Part A with good agitation while maintaining emulsion temperature at 80° C. Combined Part C ingredients and added to emulsion. Mixed continuously while cooling to 40° C. Adjusted pH between 6.0 to 6.5. Added Part D (preservative) to emulsion. Mixed well cooled and filled.

TABLE 3

| | Cationic | | | | |
|---|---|---|---|---|---|
| | hydrophobically | | Lotion | | |
| | modified | Commercial | Viscosity | | |
| | polysaccharide | Polymer and other | at 5 rpm | | |
| ID# | polymer | polymers | (cps) | pH | Comments |
| 3-1 | | Control - Polymer- | 6,800 | 6.3 | Fluid |
| | | Free | | | |
| 3-2 | | Natrosol ® Plus 330 | 124,000 | 6.2 | Smooth, Glossy, cream |
| 3-3 | | N-Hance ® 3215 | | | Phase separation |
| 3-4 | Cat C4HMGuar | | | | Separation |
| 3-5 | Cat C4HMHEC | | 12,400 | 6.24 | Fluid slightly grainy |
| 3-6 | Cat C8HMHEC | | 16,000 | 6.0 | Fluid slightly grainy |
| 3-7 | | C8HMEC | 10,800 | 6.5 | Fluid, Glossy |
| 3-8 | Cat C16HMHEC | | 110,000 | 6.4 | Creamy. Very slight grainy |
| 3-9 | Cat C16HMHEC | | 92,800 | 6.1 | Creamy. Very slight grainy |
| 3-10 | | C16HMEC | 164,000 | 6.4 | Stable, grainy, Highly |
| 3-11 | | UCARE ® LR400 | 28,000 | 6.2 | Curdled appearance. No |
| | | | | | separation |
| 3-12 | | UCARE ® JR30M | 19,200 | 6.1 | Curdled appearance. No |
| | | | | | separation |
| 3-13 | | Polysurf ® 67 | 165,000 | 6.4 | Stable, glossy, Highly |
| | | | | | structured |
| 3-14 | | Natrosol ® 250M | 5,600 | 6.3 | Fluid |
| | | | | | Glossy |
| 3-15 | | Natrosol ® 250LR | 4,400 | 6.6 | Fluid Glossy |
| 3-16 | | Natrosol ® Plus 330 | 131,000 | 6.6 | creamy Glossy |
| 3-17 | | Natrosol ® 250HHR | 12,400 | 6.5 | Fluid Glossy |
| 3-18 | | Nexton ® 3082R | 8,800 | 6.4 | Fluid Glossy |
| 3-19 | | SoftCat ® SL60 | — | | Curdled appearance. |
| | | | | | Phase separation |
| 3-20 | Cat C16HMHEC | | 22,400 | 6.4 | Grainy appearance |

Cationic hydrophobically modified polysaccharide polymer are used in Examples 3-4, 3-5, 3-6, 3-8, 3-9 and 3-20. The comparative examples are provided with Examples 3-1, 3-2, 3-3, 3-7 and 3-10 to 3-19.

The skin lotion made with Cat C16HMHECs was creamier compared to the nonionic C16HMHEC and commercial Polysurf® 67 cetyl hydroxyethyl cellulose but comparable to commercial Natrosole Plus 330CS hydroxyethyl cellulose.

Cat C8HMHEC and nonionic C8HMEC made creamy lotion but did not provide viscosity efficiency. However, still Cat C8HMHEC provided greater viscosity than C8HMHEC.

The cationic hydrophobically modified polysaccharide polymer Cat C4HMHEC was compatible in skin lotion formulation. The lotion containing commercial C4HMHEC (Nexton) was creamy. However, cationic C4HMguar was not compatible.

Commercial Cat HECs (LR400 and JR30M); Commercial Mixquat HMHEC (SoftCat® SL 60) from Dow Chemicals formed a lotion with curdled appearance and not aesthetically pleasant. Commercial Cat guar (N-Hance) 3215) failed due to phase separation.

### Ingredient List FOR TABLE 3:

### (1) Kessco® EGMS, Stepan Company, Northfield, Ill.

(2) Inustrene® 5016, Crompton Corp. Middleburry, Conn.

### (3) Drakeol® 7, Penreco, Pennzoil Products Company Karn City, Pa.

(4) Lipolan 98, Lipo Chemicals. Inc. Paterson, N.J.
(5) Crodacol® C95,: Croda Inc. Parsippany, N.J.

### (6) Germaben® II preservative from ISP Wayne, N.J.

### (7) Natrosol® Plus 330 C16 hydrophobically modified hydroxyethyl cellulose, Hercules Incorporated, Wilmington, Del.

### (8) N-Hance® 3215 cationic guar, Hercules Incorporated, Wilmington, Del.

(9) Cationic DS=0.42, Molecular weight 1.4 million. Cationic hydrophobically modified polysaccharide polymer, C4 Hydrophobically modified cationic guar, Hercules Incorporated, Wilmington, Del.
(10) Cationic hydrophobically modified polysaccharide polymer, Wt % C4HM 1.59%, cationic charge density 1.30. Cat. C4 Hydrophobically modified hydroxyethyl cellulose Hercules Incorporated, Wilmington, Del.

### (11) Cationic hydrophobically modified polysaccharide polymer, Cationic C8 hydrophobically modified hydroxyethyl cellulose from Hercules Incorporated

### (12) Cationic hydrophobically modified polysaccharide polymer, C8 hydrophobically modified hydroxyethyl cellulose from Hercules Incorporated

### (13) Cationic hydrophobically modified polysaccharide polymer, Wt % C16HM=1.48%, Cationic DS=0.08 Cationic C16 hydrophobically modified hydroxyethyl cellulose from Hercules Incorporated

### (14) Cationic hydrophobically modified polysaccharide polymer, wt % C16=1.36%, Cationic DS=0.08. Cationic C16 hydrophobically modified hydroxyethyl cellulose from Hercules Incorporated

### (15) Cationic hydrophobically modified polysaccharide polymer, C16 hydrophobically modified hydroxyethyl cellulose from Hercules Incorporated

### (16) UCARE® LR400 cationic HEC from Dow Chemicals, Midland, Mich.

### (17) UCARE® JR30M cationic HEC from Dow Chemicals, Midland, Mich.

### (18) Polysurf® 67, hydrophobically modified hydroxyethyl cellulose from Hercules Incorporated

### (19) Natrosol® 250LR hydroxyethyl cellulose from Hercules Incorporated Wilmington, Del.

### (20) Natrosol® 250M hydroxyethyl cellulose from Hercules Incorporated Wilmington, Del.

### (21) Nexton® 3082R C4 hydrophobically modified hydroxyethyl cellulose from Hercules Incorporated Wilmington, Del.

### (22) Natrosol® 250HHR CS hydroxyethyl cellulose from Hercules Incorporated Wilmington, Del.

### (23) Cationic hydrophobically modified polysaccharide polymer, Cationic DS=0.08, wt % C16HM=0.94. Cationic C16 hydrophobically modified hydroxyethyl cellulose from Hercules Incorporated Wilmington, Del.

### (24) Cationic hydrophobically modified polysaccharide polymer, cationic DS=0.08, wt % C16HM=0.92% Cationic C16 hydrophobically modified hydroxyethyl cellulose from Hercules Incorporated Wilmington, Del.

### Example 4

### Personal Care

### Body Wash

The Body wash was prepared as follows: An aqueous stock solution of each polymer was first prepared at 1.0% concentration. For guar based polymers such as: N-Hance® 3215, solutions were made by adding polymer to water under well agitation. Next, pH was lowered to between 6 to 7 with citric acid and solution was mixed for an hour or until polymer solubilized. The solutions were preserved with 0.5% Glydant® preservative. For the cellulosic polymers, the polymer was added to well agitated water and then pH was raised to 8.5 to 9.5 using sodium hydroxide. The solution was mixed for an hour and then pH was lowered to 7 using citric acid.

Body wash stock solution was prepared by adding to vessel:
46.4 grams sodium laureth sulfate,
27.0 grams sodium lauryl sulfate,
6.7 grams TEA C₁₂₋₁₃ alkyl phosphate,
4.0 grams PPG-2 hydroxyethyl Cocamide,
1.0 grams sodium chloride,
0.30 grams Tetra sodium EDTA and
0.5 grams DMDM Hydantoin in the order listed while mixing.

Each ingredient was allowed to mix homogeneously before adding the next ingredient.

The body wash stock solution batch weighed 85.9 grams.

Body wash was prepared by adding 20 grams of 1.0% solution of polymers (listed in Table 4) to 80 grams of the above body wash stock solution while mixing. Next, the body wash pH was adjusted between 6 and 7 with citric acid. The body wash viscosity was measured using the Brookfield LVT viscometer. The viscosity was measured at 30 rpm once body wash conditioned for at least two hours at 25° C. The body wash clarity was also measured at 600 nm using a Spectrophotometer, Cary 5E UV-VIS-NIR, available from Varian Instruments, Inc., or equivalent. The clarity measurements at 600 nm wavelength are reported as % T value. The higher the number, the clearer is the solution.

The body wash formulations were tested for their lather properties.

### Lather Drainage Test:

Objective of this test was to measure the lather drainage time of a diluted body wash solution. A long drainage times indicate a rich, dense lather with good stability.

### Equipment:

### Waring® Blender Model #7012, 34BL97, or equivalent.

Funnel, preferably plastic; 6″ diameter, ⅞″ ID neck, 5¼″ high, with a horizontal wire 2″ from the top. U.S.A. Standard Testing Sieve NO.20 or Tyler® Equivalent 20 mesh or 850 micrometer or 0.0331 inch sieve. Preferably, over 7 inch in diameter but smaller size could also be used.

### Stopwatch or a timer.

### Procedure:

For each test formulation, 1000 g of a diluted body wash solution was prepared as shown below.

| | |
|---|---|
| Body wash | 066.13 g |
| Deionized Water | 933.87 g |
| Total | 1000.00 g |

1. For each lather test measurement 200 grams of above diluted solution was weighed and placed in a 25° C. water-bath for 2 hours. Total of three jars each with 200 grams of solutions were prepared per body wash formulation

2. Next, the lather drainage time for each solution was measured using the procedure described below.
a. Poured 200 g of solution into a clean, dry waring blender glass vessel.
b. Whipped at the highest speed for exactly 1 minute while covered.
c. Foam generated in the jar was immediately poured into a clean, dry funnel standing on a 20 mesh screen over a beaker.
d. Foam from the blender was poured for exactly 15 seconds. The goal is to get as much foam as possible into the funnel without overflowing. After 15 seconds stopped pouring foam however, the stopwatch was kept running.
e. The total time needed for the foam to drain including the 15 seconds for pour time was recorded once the wire was no longer covered by foam or liquid.

TABLE 4

| | Cationic | | | | | |
|---|---|---|---|---|---|---|
| | hydrophobically | | | | | |
| | modified | Commercial | | Lather | | |
| | polysaccharide | Polymer and | Visc. | Stability | T | |
| ID# | polymer | other polymers | (cps) | Seconds | (%) | Comments |
| 4-1 | | Control - | 3,680 | 54 | 99.4 | |
| | | Polymer-Free | | | | |
| 4-2 | | N-Hance ® | 6,100 | 98.7 | 85.9 | |
| | | 3215 | | | | |
| 4-3 | CatC4HMGuar | | 4,380 | 79 | 94 | |
| 4-4 | CatC4HMHEC | | 4,940 | 61.3 | 99 | |
| 4-5 | Cat C16HMHEC | | 5,200 | 59 | 97.1 | |
| 4-6 | Cat C16HMHEC | | 8,820 | 57.7 | 93.8 | |
| 4-7 | | C16HMHEC | 3,960 | 57.3 | 25.2 | Applesauce like structure, |
| | | | | | | separation |
| 4-8 | | UCARE ® | 6,420 | 52.7 | 78.8 | |
| | | JR400 | | | | |
| 4-9 | | UCARE ® | 19,120 | 57.5 | 98.5 | |
| | | JR30M | | | | |
| 4-10 | | Natrosol ® Plus | 4,080 | 64.3 | 21.6 | Applesauce like structure |
| | | 330 | | | | |
| 4-11 | | Polysurf 67 | 4,080 | 52.3 | 14.2 | Applesauce like structure |
| 4-12 | | Natrosol 250M | 4,540 | Not Run | 32.4 | Gels - incompatible |
| 4-13 | | Nexton 3082R | 4,420 | 53.3 | 19.4 | |
| 4-14 | | Natrosol | 4,680 | Not run | 52.1 | Gels - Incompatible |
| | | 250HHR CS | | | | |
| 4-15 | | N-Hance HP40 | Not run | Not run | Not run | Incompatible |
| 4-16 | | Jaguar HP105 | Not run | Not run | Not run | Incompatible |
| 4-17 | Cat C16HMHEC | | 22,250 | 45.7 | 91 | Slight haze |
| 4-18 | | SoftCat | 13,120 | 45 | 98.6 | |
| | | Polymer SL-60 | | | | |
| 4-19 | | No Polymer | 7,060 | 44.7 | 99.2 | |
| | | control | | | | |
| 4-20 | | AquaCat | 7,950 | 56.8 | 99.5 | |
| | | CG518 | | | | |

The cationic hydrophobically modified polysaccharide polymer are found in examples 4-3, 4-4, 4-5, 4-6, and 4-17. The comparative examples are provided by Examples 4-1, 4-2 4-7 to 4-16, 4-18 to 4-20. Cat C16HMHEC provided better clarity and compatibility than the commercial nonionic C16HMHECs—Polysurf® 67 and Natrosol® Plus 330 and developmental C16HMHEC.

Cat C4HMEC and the Cat C4HM Guar, when used in functional compositions of this invention provided better clarity than the commercial C4HMHEC Nexton®

Clarity of functional compositions of this invention comprising cationic hydrophobically modified polysaccharide polymers was equal to commercial Cat HECs (UCARE® JR400 and JR30M) and mix quat HMHEC (Soft Cat® SL60) from Dow Chemicals.

The cationic hydrophobically modified polysaccharide polymer offered compatibility which we did not achieved with nonionic hydroxyethyl cellulose (Natrosol® 250M).

### Ingredient List for Table 4:

### (1) Sodium Lauryl sulfate—Stepanol® WAC, Stepan Company Northfield, Ill. 60093.

### (2) Sodium laureth Sulfate—Rhodapex® ES-2, Rhodia, Cranbury, N.J. 08512

### (3) Cocamidopropyl betaine—Amphosol® CA, Stepan Company Northfield, Ill. 60093.

### (4) PPG-2 Hydroxyethyl Cocamide—Promidium® CO, Uniqema, New Castle, Del.

### (5) TEA C12-13 alkyl phosphate—Arlatone MAP 230 T-60, Uniqema, New Castle, Del.

### (6) Tetra Sodium EDTA—Fisher Scientific.

(7) DMDM Hydantoin, Glydant®, Lonza Inc. Fair Lawn, N.J., USA

### (8) Sodium Chloride from Baker.

### (9) Natrosol® Plus 330-C16 Hydrophobically modified Hydroxyethyl cellulose Hercules Incorporated, Wilmington, Del.

### (10) N-Hance 3215cationic guar, Hercules Incorporated, Wilmington, Del.

(11) Cationic hydrophobically modified polysaccharide polymer, cat. C4 Hydrophobically modified guar Hercules Incorporated, Wilmington, Del.
(12) Cationic hydrophobically modified polysaccharide polymer, Cat. C4 Hydrophobically modified Hydroxyethyl cellulose Hercules Incorporated, Wilmington, Del.

### (13) Cationic hydrophobically modified polysaccharide polymer, Cationic C16 hydrophobically modified hydroxyethyl cellulose from Hercules Incorporated

### (14) Cationic hydrophobically modified polysaccharide polymer, Cationic C16 hydrophobically modified hydroxyethyl cellulose from Hercules Incorporated

### (15) Cationic hydrophobically modified polysaccharide polymer, C16 hydrophobically modified hydroxyethyl cellulose from Hercules Incorporated

### (16) UCARE® JR400: cationic HEC from Dow Chemicals, Midland, Mich.

### (17) UCARE® JR30M: X33712-76-7, cationic HEC from Dow Chemicals, Midland, Mich.

### (18) Polysurf® 67 hydrophobically modified hydroxyethyl cellulose from Hercules Incorporated

### (19) Natrosol® 250Mhydroxyethyl cellulose from Hercules Incorporated, Wilmington, Del.

### (20) Nexton® 3082Rhydrophobically modified hydroxyethyl cellulose from Hercules Incorporated, Wilmington, Del.

### (21) Natrosol 250HHR CShydroxyethyl cellulose from Hercules Incorporated, Wilmington, Del.

### (22) N-Hance® HP40hydroxypropyl guar, Hercules Incorporated, Wilmington, Del.

### (23) Jaguar® HP105hydroxypropyl guar, HP MS 0.71, Molecular weight 4,850,000. Aqueous Brookfield viscosity 3076 cp at 1.0% Rhodia; Cranbury, N.J.

(24) SoftCat® SL60: Mixed quat hydrophobically modified hydroxyethyl cellulose. The Dow Chemical Company, Midland Mich.

### (25) AquaCat® CG518 cationic guar from Hercules Incorporated.

### Example 5

### Personal Care

Sunscreen Lotion

Preparation of Sunscreen lotion: heat Drakeol® to 75° C. while mixing. Next, while mixing remaining ingredients of Part A: Arimol® E, Neo Heliopan® AV, Uvinole M40, Castor wax, Crill®-6, Arlatone® T, Ozokerite® wax and Dehymuls® HRE7 were added in the order listed. The mixture was mixed for 30 minutes at 70° C. In a separate container, water of Part B was heated to 70° C. Next, the cationic hydrophobically modified polysaccharide polymer or comparative polymer (listed in Table 5) was added and mixed until dissolved and then while mixing glycerin was added. In a separate container, solution of magnesium sulfate was prepared by adding magnesium sulfate to water and mixing for few minutes. Next, solution of magnesium sulfate was added to Part B and mixed until heated back to 70° C. This mixture was then added to Part A while mixing. Mixed for 30 minutes at 70° C. and then cooled to room temperature while mixing. Preservative Germaben® II was added when temperature reached below 50° C.

Part A

| | |
|---|---|
| 13.0 g | Drakeol 7: Mineral oil. |
| 6.0 g | Arlamol E: PPG-15 Stearyl ether |
| 1.0 g | Neo Heliopan AV: Octyl methoxcinnamate |
| 1.0 g | Uvinol M40: Benzophenone-3 |
| 1.4 g | Castor Wax: Hydrogenated castor oil |
| 1.2 g | Crill-6: Sorbitan iostearate |
| 1.0 g | Arlatone T: PPG-40 Sorbitan Peroleate |
| 1.0 g | Ozokerite Wax 77W: Wax |
| 0.5 g | Dehymuls HRE7: PEG-7 hydrogenated castor oil |

Part B

| | |
|---|---|
| 40.5 g | Deionized water |
| 0.5 g | Polymer |
| 3.0 g | Glycerine |

Part C

| | |
|---|---|
| 23.1 g | Deionized water |
| 0.7 g | Magnesium Sulfate |

Part D

| | |
|---|---|
| 0.5 g | Germaben ® II - Preservative, |

| | Cationic | | | |
|---|---|---|---|---|
| | hydrophobically | | | |
| | modified | Commercial or other | | |
| | polysaccharide | polymers | Visc. | |
| ID# | polymer | Polymer | (cps) | Comments |
| 5-1 | | Control - Polymer-Free | 4,400 | |
| 5-2 | Cat C4HMguar | | 6,380 | |
| 5-3 | Cat C4HMHEC | | 6,220 | |
| 5-4 | Cat C8HMHEC | | 6,660 | |
| 5-5 | | C8HMEC | | Phase |
| | | | | separation |
| 5-6 | Cat C16HMEC | | 5,880 | |
| 5-7 | Cat C16HMHEC | | 9,280 | |
| 5-8 | | C16HMEC | 6,060 | |
| 5-9 | | UCARE ® JR400 | 8,120 | |
| 5-10 | | UCARE ® JR30M | 3,516 | Very few |
| | | | | small |
| | | | | particles |
| | | | | at top |
| 5-11 | | Natrosol ® Plus 330 | 5,880 | |
| 5-12 | | Polysurf ® 67 | 5,260 | |
| 5-13 | | Natrosol ® 250M | 3,540 | |
| 5-14 | | Nexton ® 3082R | 5,700 | |
| 5-15 | | Natrosol ® 250HHR CS | 2,500 | |

Examples 5-2, 5-3, 5-4, 5-6 and 5-7 contain cationic hydrophobically modified polysaccharide polymers. Examples 5-1, 5-5, 5-8 to 5-15 provide comparative examples.

In the sunscreen, Cat C16HMHECs provided equal or better viscosity than the nonionic C16HMHEC Natrosol® Plus 330, Polysurf® 67).

In the sunscreen, Cat C8HMHEC was compatible while the nonionic C8HMHEC was not compatible.

In the sunscreen, Cat C4HMHEC (ADPP 5922) provided viscosity equal or better than the nonionic C4HMHEC (Nexton® 3082R).

In addition, the cationic hydrophobically modified polysaccharide polymer found in the functional compositions of this invention have affinity for skin which the conventional nonionic do not provide.

### Ingredient List FOR TABLE 5:

### (1) Drakeol® 7mineral oil, Penereco, Karn City, Pa.

### (2) Arlamol® E OOG-15 stearyl ether, Uniqema Americas, New Castle, Del.

### (3) Neo Heliopan® AV: octyl methoxcinnamate, Symrise, Totowa, N.J.

### (4) Uvinol® M40 benzophenone-3, BASF, Mount Olive, N.J.

### (5) Castor Wax hydrogenated castor oil, Frank B. Ross

(7) Crill®-6 Sorbitan iostearate, Croda Inc Parsippany, N.J.
(8) Arlatone® T PPG40 Sorbitan Peroleate, Uniqema Americas, New Castle, Del.
(9) Ozokerite® Wax 77W wax, Frank B. Ross
(10) Dehymuls® HRE7, PEG-7 hydrogenated castor oil, Cognis, Amber, Pa.
(11) Magnesium sulfate—J. T. Baker, Phillpsburg, N.J.
(12) Glycerine, Spectrum Bulk Chemicals, New Brunswick, N.J.
(13) Germaben® II preservative, ISP, Wayne, N.J.
(14) Natrosol® Plus C16 hydrophobically modified hydroxyethyl cellulose from Hercules Incorporated, Wilmington, Del.
(15) Cationic hydrophobically modified polysaccharide polymer, Wt % C4HM 1.59%, Cationic charge density 1.30. Cat. C4 Hydrophobically modified Hydroxyethyl cellulose Hercules Incorporated, Wilmington, Del.
(16) Cationic hydrophobically modified polysaccharide polymer, Cationic DS=0.029, Wt % C8=0.59%, Cationic C8 hydrophobically modified hydroxyethyl cellulose from Hercules Incorporated
(17) Cationic hydrophobically modified polysaccharide polymer, C8 hydrophobically modified hydroxyethyl cellulose from Hercules Incorporated
(18) Cationic hydrophobically modified polysaccharide polymer, wt % C16=1.48%, Cationic DS=0.08. Cationic C16 hydrophobically modified hydroxyethyl cellulose from Hercules Incorporated, Wilmington, Del.
(19) Cationic hydrophobically modified polysaccharide polymer, wt % C16=1.36%, Cationic DS=0.08. Cationic C16 hydrophobically modified hydroxyethyl cellulose from Hercules Incorporated
(20) Cationic hydrophobically modified polysaccharide polymer, C16 hydrophobically modified hydroxyethyl cellulose from Hercules Incorporated
(21) UCARE® JR400: Cationic HEC from Dow Chemicals, Midland, Mich.
(22) UCARE® JR30MCationic HEC from Dow Chemicals, Midland, Mich.
(23) Polysurf® 67 C16 hydrophobically modified hydroxyethyl cellulose from Hercules Incorporated
(24) Natrosol® 250Mhydroxyethyl cellulose from Hercules Incorporated, Wilmington, Del.
(25) Nexton® 3082R C4 hydrophobically modified hydroxyethyl cellulose from Hercules Incorporated, Wilmington, Del.
(26) Natrosol® 250HHR CShydroxyethyl cellulose from Hercules Incorporated, Wilmington, Del.

### Example 6

### Personal Care

### Roll-On Antiperspirant

Preparation: An aqueous stock solution of each polymer was first prepared at 1.0% concentration. For guar based polymers: N-Hance® 3215 solutions were made by adding polymer to water under well agitation. Next, pH was lowered to between 6 to 7 with citric acid and solution was mixed for an hour or until polymer solubilized. The solutions were preserved with 0.5% Glydant® preservative. For the cellulosic polymers, the polymer was added to well agitated water and then pH was raised to 8.5 to 9.5 using sodium hydroxide. The solution was mixed for an hour and then pH was lowered to between 6 to 7 using citric acid.

A 150 gram batch of roll-on antiperspirant was made using the procedure outlined below:

15.0 g Polymer stock solutions was added to 8 oz glass jar. Mixed with magnetic plate and stirrer. While mixing added 22.5 g deionized water. Continued mixing for about 30 minutes. While mixing added 45.0 g Ethanol. Mixed for 10 minutes. While mixing added 67.5 g antiperspirant active, Summit ACH 303. Mixing was continued for 30 more minutes.

| | Cationic | | | |
|---|---|---|---|---|
| | hydrophobically | | | |
| | modified | | | |
| | polysaccharide | Commercial or Other | Visc. | |
| ID# | polymer | polymers | (cps) | Comments |
| 6-1 | | Control - Polymer-Free | 5.5 | Clear, water-white |
| 6-2 | | N-Hance ® 3215 | Separated | Very Hazy, gels through-out |
| 6-3 | Cat C4HMGuar | | 9.6 | Trace Yellow, Slight Haze, fine |
| | | | | particles through-out |
| 6-4 | Cat C4HMHEC | | 8.8 | Sever grainy |
| 6-5 | Cat C8HMHEC | | 14.4 | Clear, trace fine particles |
| 6-6 | | C8HMHEC | 6.8 | Clear, smooth |
| 6-7 | Cat C16HMHEC | | 7.8 | Clear smooth |
| 6-8 | Cat C16HMHEC | | 8.1 | Clear, smooth |
| 6-9 | | C16HMHEC | 19.4 | Grainy |
| 6-10 | | UCARE ® JR400 | 9.9 | Clear, smooth |
| 6-11 | | UCARE ® JR30M | 31.4 | Clear smooth |
| 6-12 | | Natrosol ® Plus 330 | 11.4 | Clear, Trace haze, fine particles |
| | | | | through-out |
| 6-13 | | Polysurf ® 67 | 18.6 | Clear, fine particles through-out |
| 6-14 | | Natrosol ® 250M | 16 | Clear, water-white, fine particles |
| | | | | through-out |
| 6-15 | | Nexton ® 3082R | 36.7 | |
| 6-16 | | Natrosol ® 250HHR CS | 39 | Clear, water-white, fine particles |
| | | | | through-out |

Examples 6-3, 6-4, 6-5, 6-7 and 6-8 contain cationic hydrophobically modified polysaccharide polymers. The examples 6-1, 6-2, 6-6, 6-9 to 6-16 represent comparative examples.

In the antiperspirant, Cat C16HMHECs provided clear final product while the nonionic C16HMHEC, Natrosol® Plus 330, Polysurf® 67 some graininess was observed.

In the antiperspirant, Cat C8HMHEC provided higher viscosity than the nonionic C8HMHEC.

In the antiperspirant, Cat C4HMHEC had severe graininess than the nonionic C4HMHEC (Nexton® 3082R).

### Ingredient List FOR TABLE 6:

(1) Ethanol: dehydrated ethanol; from Spectrum Chemicals MFG Corp, Gardena, Calif.
(2) Summit® ACH-303-50% aqueous solution of aluminum chlorohydrate, from Summit Research Labs, 45 River Road, Flemington, N.J.
(3) Natrosol® Plus 330 C16 hydrophobically modified hydroxyethyl cellulose, Hercules Incorporated, Wilmington, Del.

### (4) N-Hance® 3215 cationic guar, from Hercules Incorporated, Wilmington, Del.

(5) Cationic hydrophobically modified polysaccharide polymer, cat. C4 hydrophobically modified guar, from Hercules Incorporated, Wilmington, Del.
(6) Cationic hydrophobically modified polysaccharide polymer, Cat C4 Hydrophobically modified hydroxyethyl cellulose, from Hercules Incorporated, Wilmington, Del.
(7) Cationic hydrophobically modified polysaccharide polymer, cationic C8 hydrophobically modified hydroxyethyl cellulose from Hercules Incorporated, Wilmington, Del.
(8) Cationic hydrophobically modified polysaccharide polymer, C8 hydrophobically modified hydroxyethyl cellulose from Hercules Incorporated, Wilmington, Del.
(9) Cationic hydrophobically modified polysaccharide polymer, cationic C16 hydrophobically modified hydroxyethyl cellulose from Hercules Incorporated, Wilmington, Del.
(10) Cationic hydrophobically modified polysaccharide polymer, cationic C16 hydrophobically modified hydroxyethyl cellulose from Hercules Incorporated, Wilmington, Del.
(11) 1 Cationic hydrophobically modified polysaccharide polymer, C16 hydrophobically modified hydroxyethyl cellulose from Hercules Incorporated, Wilmington, Del.
(15) UCARE® JR400 cationic hydroxyethyl cellulose from Dow Chemicals, Midland, Mich.
(16) UCARE® JR30M cationic hydroxyethyl cellulose from Dow Chemicals, Midland, Mich.
(17) Polysurf® 67hydrophobically modified hydroxyethyl cellulose from Hercules Incorporated, Wilmington, Del.
(18) Natrosol® 250M hydroxyethyl cellulose from Hercules Incorporated, Wilmington, Del.
(19) Nexton® 3082R hydrophobically modified hydroxyethyl cellulose from Hercules Incorporated, Wilmington, Del.
(23) Natrosol® 250HHR CShydroxyethyl cellulose from Hercules Incorporated, Wilmington, Del.

### Example 7

### Household & Institutional

### All-Purpose Cleaner

Twenty grams of 1.0% polymer solution was added to 80 grams of Lysol® All-Purpose Cleaner, available from Reckitt Benckiser plcand mixed for few minutes. The formulation stability was checked after two week and after about two months. The formulation data are provided in Table 7.

Polymer Solution preparation: 1.00% stock solutions of Natrosol® 250HHR CS and Cellulosic polymers of invention were prepared by adding 4.0 g of polymer to 396.0 g of water under agitation. The solution pH was adjusted to above 7 with 1.0% NaOH solution if required. The solution was mixed for 45 minutes and then pH was lowered below 7. As a preservative 2.0 g Glydant® was added. Solutions of guar-based products were made by adding 4.0 g of polymer to 396.0 g of water under agitation. The solution pH was adjusted to below 7 with citric acid solution if required. The solution was mixed for 45 minutes. As a preservative 2.0 g Glydant® preservative was added. To 80.0 g of products, 20.0 g of polymer solution was added. The solution was shaken by hand and then tumbled for 1.5 hours on tumbler. Final product viscosity was measured using the Brookfield LVT viscometer once the product was conditioned for at least two hours at 25° C. The product clarity was also measured at 600 nm using a Spectrophotometer, Cary 5E UV-VIS-NIR, available from Varian Instruments, Inc., or equivalent. The clarity measurements at 600 nm wavelength are reported as % T value. The higher the number, the clearer is the solution. Viscosity, pH and % T were measured by after two weeks. Visual observations were also made after two weeks and after about two months.

TABLE 7

| | Cationic | | | | | |
|---|---|---|---|---|---|---|
| | hydrophobically | Commercial | | | | |
| | modified polysaccharide | Polymer or other | | Viscosity | | Two month |
| ID# | polymer | polymers | pH | (cps) | % t | stability |
| 7-1 | | Lysol ® All- | 8.8 | 3.5 | 99.3 | Clear |
| | | Purpose Cleaner, | | | | |
| | | as received | | | | |
| 7-2 | | 80/20 Lysol/Water | 8.8 | 3.2 | 99.2 | Clear |
| 7-3 | | N-Hance ® 3215 | | 8.4 | 99.5 | Not Stable |
| | | cationic guar | | | | |
| 7-4 | Cat C4HMguar | | 8.6 | 6.4 | 98.7 | Not stable |
| 7-5 | Cat C4HMHEC | | 8.6 | 8.6 | 99.8 | Stable |
| 7-6 | | C8HMHEC | 8.7 | 4.4 | 99.7 | Stable |
| 7-7 | CatC8HMHEC | | 8.6 | 5.4 | 99.5 | Stable |
| 7-8 | | C16HMHEC | 8.6 | 11.3 | 98.5 | Stable |
| 7-9 | | Polysurf 67 | 8.6 | 12.4 | 99.6 | Stable |
| 7-10 | | Natrosol ® Plus | 8.6 | 6 | 99.8 | Stable |
| | | 330 HMHEC | | | | |
| 7-11 | Cat C16HMHEC | | 8.6 | 6.5 | 99.9 | Stable |
| 7-12 | Cat C16HMHEC | | 8.5 | 4.6 | 94.8 | Stable |
| 7-13 | | Natrosol ® | 8.6 | 19.1 | 99.9 | Stable |
| | | 250HHR HEC | | | | |
| 7-14 | | Cat HEC, UCARE | 8.6 | 6.8 | 99.4 | Stable |
| | | JR400 | | | | |
| 7-15 | | Cat HEC UCARE | 8.7 | 13.1 | 100 | Stable |
| | | JR30M | | | | |

Examples 7-4, 7-5, 7-7, 7-11 and 7-12 contain cationic hydrophobically modified polysaccharide polymers. The examples 7-1 to 7-3, 7-6, 7-8 to 7-10 and 7-13 to 7-15 represents comparative examples.

In Lysol® Disinfectant Household & Institutional cleaner Table 7, both nonionic and cationic HMHEC provided clarity and compatibility comparable to the control without the polymer. The cationic offers affinity for anionic surface, which nonionic would not.

### Ingredient List FOR TABLE 7:

(1) Natrosol® Plus, C16 Hydrophobically modified Hydroxyethyl cellulose Hercules Incorporated, Wilmington, Del.
(2) N-Hance® 3215, cationic guar, Hercules Incorporated, Wilmington, Del.
(3) Cationic hydrophobically modified polysaccharide polymer, cat. C4 Hydrophobically modified guar Hercules Incorporated, Wilmington, Del.
(4) Cationic hydrophobically modified polysaccharide polymer, Cat. C4 Hydrophobically modified Hydroxyethyl cellulose Hercules Incorporated, Wilmington, Del.
(5) Cationic hydrophobically modified polysaccharide polymer, Cationic C8 hydrophobically modified hydroxyethyl cellulose from Hercules Incorporated
(6) Cationic hydrophobically modified polysaccharide polymer, CB hydrophobically modified hydroxyethyl cellulose from Hercules Incorporated, Wilmington, Del.
(7) Cationic hydrophobically modified polysaccharide polymer, Cationic C16 hydrophobically modified hydroxyethyl cellulose from Hercules Incorporated, Wilmington, Del.
(8) Cationic hydrophobically modified polysaccharide polymer, Cationic C16 hydrophobically modified hydroxyethyl cellulose from Hercules Incorporated, Wilmington, Del.
(9) Cationic hydrophobically modified polysaccharide polymer, C16 hydrophobically modified hydroxyethyl cellulose from Hercules Incorporated, Wilmington, Del.
(10) UCARE® JR400, cationic HEC from Dow Chemicals, Midland, Mich.
(11) UCARE® JR30Mcationic HEC from Dow Chemicals, Midland, Mich.
(12) Polysurf® 67, hydrophobically modified hydroxyethyl cellulose from Hercules Incorporated, Wilmington, Del.
(13) Natrosol® 250HHR CS, hydroxyethyl cellulose from Hercules Incorporated, Wilmington, Del.

### Example 8

### Hand Soap Household & Institutional

### Hand Soap:

A commercial liquid hand soap was used for the study. Twenty grams of 1.0% polymer solution was added to 80 grams of liquid hand soap, Softsoap® liquid hand soap, available from Colgate-Palmolive Company, and mixed for few minutes. The formulation stability was checked after two week and after about two months. The formulation data are provided in Table 8

TABLE 8

| | Cationic | | | | | |
|---|---|---|---|---|---|---|
| | hydrophobically | | | | | |
| | modified | | | | | |
| | polysaccharide | Commercial or Other | | | | Two month |
| ID# | polymer | Polymer | pH | Viscosity | % t | stability |
| 8-1 | | Softsoap ® liquid hand soap, | 7.2 | 4600 | 97.5 | Stable, Clear |
| | | as received | | | | |
| 8-2 | | 80/20 Softsoap ® liquid hand | 7.2 | 173 | 97.1 | Stable, Clear |
| | | soap | | | | |
| 8-3 | | N-Hance ® 3215 cationic | 7.2 | 650 | 54.2 | Stable, Hazy |
| | | guar | | | | |
| 8-4 | Cat C4HMguar | | 7.2 | 288 | 6.2 | Unstable |
| 8-5 | Cat C4HMHEC | | 7.1 | 384 | 67.3 | Stable, Hazy |
| 8-6 | | C8HMHEC | 7.2 | 331 | 96.8 | Stable, Clear |
| 8-7 | CatC8HMHEC | | 7.2 | 270 | 86.5 | Stable Slightly Hazy |
| 8-8 | | C16HMHEC | 7.2 | 1736 | 87.5 | Stable Slightly Hazy |
| 8-9 | | Polysurf ® 67 HMHEC | 7.2 | 1380 | 40.4 | Unstable |
| 8-10 | | Natrosol ® Plus 330 HMHEC | 7.2 | 774 | 81.2 | Unstable |
| 8-11 | Cat C16HMHEC | | 7.2 | 2640 | 97.6 | Stable Clear |
| 8-12 | Cat C16HMHEC | | 7.2 | 2572 | 97.1 | Stable Clear |
| 8-13 | | Natrosol ® 250HHR HEC | 7.2 | 282 | 74.1 | Unstable |
| 8-14 | | Natrosol ® 250M HEC | 7.2 | 282 | 46.6 | Unstable |
| 8-15 | | Cat HEC, UCARE JR400 | 7.2 | 249 | 32.1 | Stable Very Hazy |
| 8-16 | | Cat HEC UCARE JR30M | 7.2 | 665 | 94.5 | Stable Slightly hazy |

Examples 8-4, 8-5, 8-7, 8-11, 8-12 contain cationic hydrophobically modified polysaccharide polymers. The examples 8-1 to 8-3, 8-6, 8-8, to 8-10, 8-13 to 8-16 represents comparative examples.

In Softsoap® hand soap Table 8, Cat C4HMHEC was compatible but soap became hazy. The Cat C8HMHEC was also compatible but the soap was hazy. The Cat C16HMHECs were not only compatible but also retained clarity of the original Softsoap® hand soap. The soap was either hazy or incompatible with nonionic C16HMHECs. In addition, the Cat C16HMHEC provided much higher viscosity than the nonionic C16HMHECs. That is manufacturer can reduce the level of surfactant in the liquid soap and further improve mildness of the hand soap. The nonionic HEC (Natrosol®) were incompatible with Softsoap® handsoap. The commercial Cat HEC (UCARE JR400, JR30M) made the Softsoap® handsoap hazy.

Ingredient List FOR TABLE 8:
(1) Natrosol® Plus 330 C16 hydrophobically modified Hydroxyethyl cellulose Hercules Incorporated, Wilmington, Del.
(2) N-Hance® 3215cationic guar, Hercules Incorporated, Wilmington, Del.
(3) Cationic hydrophobically modified polysaccharide polymer, cat. C4 Hydrophobically modified guar, Hercules Incorporated, Wilmington, Del.
(4) Cationic hydrophobically modified polysaccharide polymer, Cat. C4 Hydrophobically modified hydroxyethyl cellulose Hercules Incorporated, Wilmington, Del.
(5) Cationic hydrophobically modified polysaccharide polymer, cationic C8hydrophobically modified hydroxyethyl cellulose from Hercules Incorporated, Wilmington, Del.
(6) Cationic hydrophobically modified polysaccharide polymer, C8hydrophobically modified hydroxyethyl cellulose from Hercules Incorporated, Wilmington, Del.
(7) Cationic hydrophobically modified polysaccharide polymer, cationic C16 hydrophobically modified hydroxyethyl cellulose from Hercules Incorporated, Wilmington, Del.
(8) Cationic hydrophobically modified polysaccharide polymer, cationic C16 hydrophobically modified hydroxyethyl cellulose from Hercules Incorporated, Wilmington, Del.
(9) Cationic hydrophobically modified polysaccharide polymer, C16 hydrophobically modified hydroxyethyl cellulose from Hercules Incorporated, Wilmington, Del.
(10) UCARE® JR400 cationic HEC from Dow Chemicals, Midland, Mich.
(11) UCARE® JR30M cationic HEC from Dow Chemicals, Midland, Mich.
(12) Polysurf® 67 hydrophobically modified hydroxyethyl cellulose from Hercules Incorporated, Wilmington, Del.
(13) Natrosol® 250Mhydroxyethyl cellulose from Hercules Incorporated, Wilmington, Del.
(14) Natrosol® 250HHR CS hydroxyethyl cellulose from Hercules Incorporated, Wilmington, Del.

### Example 9

### Hand Soap Household & Institutional

A commercial liquid hand soap, Softsoap® liquid antibacterial hand soap, available from Colgate-Palmolive Company, was used for the study. 0.2% polymer was added to 100 grams of liquid hand soap and mixed. The Softsoap® liquid antibacterial hand soap contains: water, sodium laureth sulfate, cocamidopropyl betaine, sodium chloride, decyl glucose, fragrance, DMDM hydantoin, PEG-120 methyl glucose dioleate, tetra sodium EDTA, citric acid, sodium sulfate, polyquternium-7, polaxamer 124, PEG-7 glyceryl cocoate, D&C Red NO. 33, FD&C Blue NO.1.

In another experiment, another commercially available liquid hand soap Dial® antibacterial hand soap, available from the Dial Corporation, was used for the study. Again, 0.2% polymer was added to 10 g of Dial® antibacterial hand soap and mixed. The Dial® hand soap contained water, sodium laureth sulfate, ammonium lauryl sulfate, decyl glucose, cocamidopropyl betaine, glycerin, sodium chloride, PEG-18 glyceryl oleate/cocoate, fragrance, cocamide MEA, DMDM hydantoin, tetrasodium EDTA, yellow 5, red 4.

TABLE 9

| | Cationic | | | | | |
|---|---|---|---|---|---|---|
| | hydrophobically | | | | | |
| | modified | | | | | |
| | polysaccharide | Commercial or | | Viscosity | | |
| ID# | polymer | Other Polymer | pH | (cps) | % t | Appearance |
| 9-1 | | Softsoap ® liquid hand | 7.0 | 4,580 | 95.8 | Stable, Clear |
| | | soap, as received | | | | |
| 9-2 | | Polysurf ® 67 HMHEC | 7.2 | 10,200 | 32.4 | Very Hazy, grainy |
| 9-3 | | Natrosol ® Plus 330 | 7.3 | 8,880 | 58.7 | Very Hazy, Grainy |
| | | HMHEC | | | | |
| 9-4 | Cat C16HMHEC | | Not | 22,850 | 93.5 | Clear |
| | | | Determined | | | |

Dial ® Hand Soap

| | | | | | | |
|---|---|---|---|---|---|---|
| 9.6 | | Dial ® hand soap, as | 5.9 | 3,160 | 98.7 | Clear |
| | | received | | | | |
| 9-7 | | Polysurf ® 67 HMHEC | 5.9 | 7,440 | 63.3 | Hazy, Grainy |
| 9-8 | | Natrosol ® Plus 330 | 5.8 | 6,780 | 82.2 | Hazy, grainy |
| | | HMHEC | | | | |
| 9-9 | Cat C16HMHEC | | Not | 10,850 | 96.8 | |
| | | | Determined | | | |

Example 9-4 and 9-9 contains cationic hydrophobically modified polysaccharide polymers. The addition of cationic hydrophobically modified polysaccharide polymers improves viscosity of the Softsoap® liquid hand soap and Dial/antibacterial hand soap without negatively affecting the clarity of the product. This would allow the formulator to reduce the level of surfactant and market a milder formulation while maintaining lather properties and adding conditioning properties which one does not achieve from surfactant. The nonionic commercial HMHECs made the Softsoap® liquid hand soap hazy and grainy looking. Similarly, the nonionic commercial HMHECs made Dial® antibacterial hand soap hazy and grainy.

In a subjective evaluation test, a test member preferred Softsoap® liquid hand soap and the Dial® antibacterial hand soap with cationic hydrophobically modified polysaccharide polymer over the commercial hand soap for its reach fine lather and conditioning feel.

### Ingredient List FOR TABLE 9:

(1) Natrosol® Plus 330 C16 hydrophobically modified hydroxyethyl cellulose Hercules Incorporated, Wilmington, Del.
(2) Cationic hydrophobically modified polysaccharide polymer, cationic C16 hydrophobically modified hydroxyethyl cellulose from Hercules Incorporated, Wilmington, Del.
(3) Polysurf® 67 hydrophobically modified hydroxyethyl cellulose from Hercules Incorporated, Wilmington, Del.

### Example 10

### Conditioning Shampoos

A cationic hydrophobically modified polysaccharide polymer of use in formulations of the present invention, Cat C16HMHEC, containing 1.3-1.6 wt % of C16 hydrophobe and low degree of cationic substitution was produced (Example 10). In this example the Cat HMHEC polymer was evaluated for its performance as a conditioning polymer in hair care and was compared against N-Hance® 3196 cationic guar, available from Hercules Incorporated and cellulose ether derivatives containing quaternary ammonium groups LR 400 cationic HEC, available from Union Carbide Corporation, Softcat® SL60 cationic HMHEC, available from The Dow Chemical Company and a control with no polymer.

Two shampoo formulations: Shampoo Stock Solution 1 (thickened with Natrosol® 250 HR HEC) and Shampoo Stock Solution 2 (thickened with NH₃Cl) were tested. Asian virgin, Asian bleached, Caucasian virgin and Caucasian bleached hair tresses were used in this example.

The example was carried out along two experimental approaches: wet/dry combing evaluations and the study of silicone deposition. Wet/Dry combing was carried out in formulations that were silicone free to establish conditioning properties of polymers alone. Silicone deposition was studied in the same formulations with added 3 wt % (as is) Dow Corning 1784 silicone emulsion.

Manual combing was carried out with close attention paid to hair lubricity/squeakiness and the degree of knot and tangle formation as a result of washing/combing. It should be pointed out that ‘squeakiness’ of hair is the result of stick and slip of comb over small length scale as the comb travels down the tress.

Lubricity and entanglement were rated separately on the scale from 1 to 3 as defined below:
Rating 1: very squeaky; many entanglements and knots starting at the middle of a tress and continuing to the end of a tress,
Rating 2: small but noticeable improvement over Rating 1, i.e. less squeakiness; lower degree of entanglement, i.e. smaller knots and/or knots observed only in the lower portion of the tress,
Rating 3: significant improvement over Rating 1, i.e. more lubricious feel; fewer knots.

### Shampoo Preparation

Two stock shampoo solutions Solution 1 and Solution 2 were prepared as shown in Tables 10 and 11. The shampoo stock solutions contain a hold out that allows addition of silicone emulsion (Dow Corning 1784 silicone emulsion) and/or 1 wt % of stock solutions of polymers to achieve final shampoo compositions with substantially identical content of ingredients. All final shampoos contained either 0.2 wt % of conditioning polymer or a combination of 0.2 wt % polymer and 3 wt % (as is) Dow Corning 1784 emulsion. Table 12 identifies conditioning polymers.

TABLE 10
Shampoo Stock Solution 1

| | | % | | Amount |
|---|---|---|---|---|
| Ingredient | INCI Name | Active | % AS IS | (gm) |
| Deionized water | Water | | 53.36 | 1332.75 |
| Texapon N70 NA (70%) | Sodium laureth sulfate (2EO) | 12.00 | 17.14 | 428.57 |
| Velvetex BA35 (35%) | Cocamidopropyl betaine | 1.05 | 3.00 | 75.00 |
| Comperlan COD (80%) | Coconut Fatty Acid | 1.60 | 2.00 | 50.00 |
| | Diethanolamide | | | |
| Natrosol 250 HR CS HEC | Hydroxyethylcellulose | 1.00 | 1.00 | 26.18 |
| (% M = 4.7) | | | | |
| Glydant | DMDM Hydantoin | 0.50 | 0.40 | 9.99 |

TABLE 11
Shampoo Stock Solution 2

| | INCI | % | % | Amount, |
|---|---|---|---|---|
| Ingredient | Name | Active | AS IS | (gm) |
| Deionized water | Water | | 27.65 | 691.30 |
| Steapnol AM, 28.5% actives | ALS | 9.00 | 31.58 | 789.47 |
| Steol CA 330, 28% actives | ALES | 2.67 | 9.54 | 238.39 |
| Amphosol CA, 30% actives | CAPB | 1.72 | 5.73 | 143.33 |
| Glydant | DMDM | 0.50 | 0.40 | 9.99 |
| | Hydantoin | | | |
| Salt, Amonium chloride, 25% | | 0.5 | 2.00 | 50.00 |
| | | | | 1872.49 |

TABLE 12
Conditioning polymers stock solutions

| | | MOIS- | POLY- | WA- | |
|---|---|---|---|---|---|
| | | TURE % | MERS | TER | GLYDANT |
| 12-1A | NO POLYMER | 0.00 | 1.60 | 158.40 | 0.80 |
| 12-2A | N-Hance ® 3196 | 10.03 | 1.76 | 158.24 | 0.80 |
| | cationic guar | | | | |
| 12-3A | LR 400 cationic | 6.41 | 1.70 | 158.30 | 0.80 |
| | HEC | | | | |
| 12-4A | Softcat ® SL60 | 6.23 | 1.71 | 158.29 | 0.80 |
| | cationic HMHEC | | | | |
| 12-5A | Example 10 | 3.63 | 1.66 | 158.34 | 0.80 |

### Hair Tress Preparation and Testing

Tress Pre-Treatment

Hair tresses were supplied by International Hair Importers and Products Inc, Lots: Asian virgin, Asian bleached, Caucasian virgin, Caucasian bleached. Four sets, each comprising of thirty tresses, were selected and labeled. Each tress was pretreated with 4.5% sodium laureth sulfate (SLS) wash.

Tress Preparation for Wet/Dry Combing

Wet/Dry combing was carried out on Caucasian virgin, Caucasian bleached, Asian virgin and Asian bleached hair tresses per each shampoo type without duplication. Each tress washed with corresponding shampoo. This was immediately followed by combing the tress 3 times with the large comb teeth and then 3 times with the small comb teeth. Observations such as squeakiness and the occurrence of knots/tangles were recorded. Following wet combing, the tresses were hung to dry overnight in a controlled temperature room. Dry tresses were combed the next day 3 times with the large comb teeth and then 3 times with the small comb teeth and the ease of combing recorded. All experimental work was carried out in controlled temperature room.

Silicone Deposition

The silicone deposition was carried out in duplicates by washing tresses with silicone containing shampoos. The tresses were hung to air dry overnight and then individually packaged in plastic bags.

Wet Combing

### Tables 13 and 14 show the results of wet combing after treatment with Shampoo Stock Solution 1 and Shampoo Stock Solution 2, respectively.

In the Shampoo Stock Solution 1 based formulas, the Softcat® SL60 cationic HMHEC left the Caucasian bleached and virgin hair with the least amount of tangles but a squeaky feel. The LR400 cationic HEC gave the Caucasian bleached and virgin hair the smoothest feel but left more tangles than the Softcat® SL60 cationic HMHEC. On the Asian bleached and virgin hair, Example 10 performed the best among all the polymers. The polymer left little tangles and a smooth feel.

In Shampoo Stock Solution 2 based formulas, all of the Caucasian bleached tresses had a squeaky feel. The SL60 left the Caucasian bleached tress with the least amount of tangles. Example 10 resulted in the smoothest feel on Asian bleached and virgin hair. Example 10 and Softcat® SL60 cationic HMHEC were equally best in decreasing entanglement in Asian virgin and bleached hair.

Note that Asian hair, both bleached and virgin, tend to entangle less than its Caucasian counterpart, hence, the rating 2 in No Polymer control. The improvement was attributed to higher stiffness of Asian hair that prevents knot formation during hair washing and combing.

A limited wet combing study was carried out on Asian tresses using the silicone containing Shampoo Stock Solution 1. Combing improved in all tresses making differentiation more difficult. N-Hance® 3196 cationic guar and Example 10 were slightly better on Asian bleached tresses. On the Asian virgin tresses all the polymer containing shampoos had the same effect: they were all slightly smoother than the no polymer control. All tresses had very little tangles.

TABLE 13
Wet combing results for hair treated with Shampoo Stock Solution
1 based shampoos as a function of conditioning polymer

| | | N-Hance ® | | Softcat ® | |
|---|---|---|---|---|---|
| | | 3196 | LR 400 | SL60 | Exam- |
| LUBRICY/ | No | cationic | cationic | cationic | ple |
| ENTAGLMENT | Polymer | guar | HEC | HMHEC | 10 |
| Cauc./Bleached | 1/1 | 1/1 | 2/1 | 1/2 | 1/1 |
| Cauc./Virgin | 1/1 | 1/1 | 2/1 | 1/2 | 1/2 |
| Asian/Bleached | 1/2 | 1/3 | 1/3 | 1/2 | 3/3 |
| Asian/Virgin | 1/2 | 1/3 | 1/3 | 1/2 | 3/3 |

TABLE 14
Wet combing results for hair treated with Shampoo Stock Solution
2 based shampoos as a function of conditioning polymer

| | | N-Hance ® | | Softcat ® | |
|---|---|---|---|---|---|
| | | 3196 | LR 400 | SL60 | Exam- |
| LUBRICY/ | No | cationic | cationic | cationic | ple |
| ENTAGLMENT | Polymer | guar | HEC | HMHEC | 10 |
| Cauc./Bleached | 1/1 | 2/1 | 2/1 | 1/3 | 2/2 |
| Cauc./Virgin | 1/1 | 1/1 | 2/1 | 2/2 | 2/2 |
| Asian/Bleached | ½ | 1/2 | 1/2 | 2/3 | 3/3 |
| Asian/Virgin | ½ | 1/2 | 1/2 | 2/3 | 3/3 |

Dry Combing

Dry combing produces less resistance than its wet counterpart making manual evaluation more difficult. Attempts to determine conditioning effects of the polymers in dry combing did not produce discernable results.

It is not intended that the examples presented here should be construed to limit the invention, but rather they are submitted to illustrate some of the specific embodiments of the invention. Various modifications and variations of the present invention can be made without departing from the scope of the appended claims.

What is claimed:

1. A functional composition comprising, a) at least about 0.01 wt % of a cationic hydrophobically modified polysaccharide wherein i) the cationic hydrophobic polysaccharide has a mean average molecular weight (Mw) from about 2000 to about 10,000,000 Dalton, ii) the cationic hydrophobically modified polysaccharide has a cationic degree of substitution greater than 0.001 to about 3.0, and iii) cationic hydrophobically modified polysaccharide has weight % hydrophobe greater than 0.001. b) less than 50.0 wt % total of a personal care, pet care or household & institutional care active ingredient material.

2. The functional composition of claim 1, wherein the functional composition comprises a personal care active ingredient and wherein the functional composition provides cleansing, cleaning protecting, benefiting, depositing, moisturizing, firming, conditioning, lather, barrier, and emolliency to skin and hair.

3. The functional composition of claim 1, wherein the functional composition comprises a household & institutional active ingredient and wherein the functional composition provides cleansing, cleaning protecting, benefiting, lather, depositing and barrier to a substrate.

4. The functional composition of claim 1, wherein the functional composition comprises a pet care active ingredient and wherein the functional composition provides cleansing, cleaning protecting, benefiting, depositing, moisturizing, firming, lather conditioning, barrier, and emolliency to skin and hair.

5. The functional composition of claim 2, wherein the personal care active ingredient is selected from the group consisting of uv absorbers, sun screen agents, abrasive, cleansing, cleaning, moisturizers, humectants, occlusive agents, moisture barriers, lubricants, emollients, anti-aging agents, antistatic agents, secondary conditioners, exfolliants, lustering agents, tanning agents, luminescents agents, colors, anti-odorizing agents, fragrances, viscosifying agents, salts, lipids, phospholipids, hydrophobic plant extracts, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons and mixtures thereof.

6. The functional composition of claim 3, wherein the household & institutional active ingredient is selected from the group consisting of uv absorbers, sun screen agents, abrasive, cleansing, cleaning, occlusive agents, moisture barriers, lubricants, emollients, anti-aging agents, antistatic agents, secondary conditioners, exfolliants, lustering agents, luminescents agents, colors, anti-odorizing agents, fragrances, viscosifying agents, salts, lipids, phospholipids, hydrophobic plant extracts, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons and mixtures thereof.

7. The functional composition of claim 4, wherein the pet care active ingredient is selected from the group consisting of uv absorbers, sun screen agents, abrasive, cleansing, cleaning, moisturizers, humectants, occlusive agents, moisture barriers, lubricants, emollients, anti-aging agents, antistatic agents, secondary conditioners, exfolliants, lustering agents, tanning agents, luminescents agents, colors, anti-odorizing agents, fragrances, viscosifying agents, salts, lipids, phospholipids, hydrophobic plant extracts, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons and mixtures thereof.

8. The functional composition of claim 1 wherein the functional composition exhibits functional properties of a conventional functional compositions without the cationic hydrophobically modified polysaccharide but at levels of active ingredient material below those of the conventional functional composition.

9. The functional composition of claim 8 wherein the functional property comprises a surfactant property.

10. The functional composition of claim 1, wherein the total amount of active ingredient material found in the functional composition is in the range of from 0.01 to 50-wt %.

11. The functional composition of claim 10, wherein the total amount of active ingredient material found in the functional composition is in the range of from 0.01 to 30 wt %.

12. The functional composition of claim 11, wherein the total amount of active ingredient material found in the functional composition is in the range of from 0.5 to 15 wt %.

13. The functional composition of claim 1, wherein the total amount of active ingredient material found in the functional composition is less than 30 wt %.

14. The functional composition of claim 13, wherein the total amount of active ingredient material found in the functional composition is less than 1 wt %.

15. The functional composition of claim 1, wherein the cationic hydrophobic polysaccharide has a mean average molecular weight (Mw) from about 50,000 to about 2,000,000. Dalton.

16. The functional composition of claim 15, wherein the cationic hydrophobic polysaccharide has a mean average molecular weight (Mw) from about 200,000 to about 1,000,000 Dalton.

17. The functional composition of claim 1, wherein the cationic hydrophobic polysaccharide has a cationic degree of substitution greater than 0.05 to about 2.0.

18. The functional composition of claim 17, wherein the cationic hydrophobic polysaccharide has a cationic degree of substitution greater than 0.1 to about 1.5.

19. The functional composition of claim 18, wherein the cationic hydrophobic polysaccharide has a cationic degree of substitution greater than 0.2 to about 1.0.

20. The functional composition of claim 1, wherein the cationic hydrophobic polysaccharide has a weight % hydrophobe in the range of 0.1 to about 5%.

21. The functional composition of claim 20, wherein the cationic hydrophobic polysaccharide has a weight % hydrophobe in the range of 0.2 to about 3%.
